(19) United States
(12) Patent Application Publication
(10) Pub. No.: US 2025/0255529 A1
(43) Pub. Date: Aug. 14, 2025

(54) MULTI-ELECTRODE CATHETER

(71) Applicant: IMPERIAL COLLEGE INNOVATIONS LTD., London (GB)

(72) Inventors: Prapa Kanagaratnam, London (GB); Nicholas Linton, London (GB); Burak Temelkuran, London (GB); Hang Xu, London (GB); Mohamed E.M.K. Abdelaziz, London (GB); Minh-Son Pham, London (GB); Li-Liang Wang, London (GB)

(21) Appl. No.: 18/856,767

(22) PCT Filed: Apr. 13, 2023
(86) PCT No.: PCT/GB2023/050989

§ 371 Date: Oct. 14, 2024

(30) Foreign Application Priority Data
Apr. 14, 2022 (GB) 2205559.4

(51) Int. Cl. A61B 5/287; A61B 5/00; A61B 5/367

(52) U.S. Cl. CPC A61B 5/287; A61B 5/367; A61B 5/6858; A61B 2562/125

(57) ABSTRACT

A multi-electrode surgical probe comprising a basket 10, and a plurality of electrodes supported on the basket, wherein the basket is flexible between a contracted configuration and a deployed configuration, and the probe comprises a bi-stable structure 12 which is stable in both the deployed configuration and the contracted configuration.

### CROSS-REFERENCE TO RELATED APPLICATIONS

This application is the National 1 Stage of International Application No. PCT/GB2023/050989, filed Apr. 13, 2023 which claims priority to GB 2205559.4 filed Apr. 14, 2022, both of which are hereby incorporated in their entireties by reference.

### FIELD OF THE INVENTION

The present invention relates to multi-electrode probes or catheters, in particular for the detection and recordal of electrical signals from the surface of the heart.

### BACKGROUND TO THE INVENTION

Atrial fibrillation (AF) counts amongst the most common heart rhythm disturbances. It is caused by abnormal areas of the heart causing disruption to the normal electrical beats in the heart. These areas can be treated by ‘ablation’—a procedure in which parts of the heart are cauterised. In order to locate the areas causing atrial fibrillation it is known to collect electrical information directly from the surface of the heart. Typically this is done using a multi-electrode catheter. Such catheters require design and manufacturing solutions which can present all of the wires to the heart surface and achieve contact at the same. The wires have to be kept fully insulated and connected outside the body to an amplifier system and computer to perform the required analysis.

A multi-electrode mapping catheter for endocardial contact mapping of a heart chamber typically includes an expandable framework or basket. Known basket designs can generally be defined in two distinct categories: pre-shaped and post-shaped. A pre-shaped basket is movable between a stressed contracted configuration and a pre-shaped deployed configuration. An example of such a catheter is described in WO2016039824A1. In contrast, a post-shaped basket is movable between a stress-free contracted configuration and a deployed configuration that is post-shaped via an external force. An example of such a catheter is described in WO2005112813A1. The pre-shaped basket has a precisely defined deployed shape but fewer wires than the post-shaped basket. An excess of wires concentrates high stress in contracted configuration and leads to high intertwined risks. A post-shaped basket can have many more wires than a pre-shaped one. However existing post-shaped baskets are deployed via large nonlinear wire bending, which requires a rigorously controlled and constantly applied load to keep their expanded shape, and the expanded shape cannot be precisely estimated or accurately controlled onsite. This uncertainty in the deformation, and hence the expanded shape, has a detrimental influence on the signal stability and the mapping accuracy.

### SUMMARY OF THE INVENTION

The present invention provides multi-electrode catheter comprising a basket supporting a plurality of electrodes wherein the basket is flexible between a deployed configuration and a contracted configuration, wherein the probe comprises a bi-stable structure which is stable in both the deployed configuration and the contracted configuration.

The basket may comprise a plurality of flexible electrode support members. Each of the electrode support members may support at least one of the electrodes. The electrode support members may be arranged to flex between a first shape and a second shape as the basket flexes between the contracted configuration and the deployed configuration. The first shape of the electrode support members may be straight and the second shape may be curved.

The electrode support members may form a hollow tubular structure when the probe is in the contracted configuration.

The basket may have a first end and a second end. The probe may further comprise first and second supports each supporting a respective end of the basket. Each of the supports may be tubular defining an opening therethrough. The opening may form an instrument channel through which an instrument can be moved through the probe. The first support may be movable relative to the second support thereby to flex the basket between the contracted configuration and the deployed configuration.

The probe may comprise a bistable member which is arranged to resist movement of the probe out of both the contracted configuration and the deployed configuration. The bistable member may be flexible. The bistable member may have a folded state and an unfolded state. The bistable member may comprise first and second main portions and a hinge portion connecting the first and second main portions to each other. The bistable member may further comprise a support portion and a further hinge portion connecting the support portion to the second main portion. The bistable member may or may not comprise one of the electrode support members.

One end of the first main portion may be supported in a first support. The support portion may be supported in a second support. The first and second supports may be moveable relative to each other to move the bistable member between its folded and unfolded states.

The bistable structure may comprise a plurality of flexible bistable members.

When the probe is in the contracted state, the bistable member or members may be parallel to the electrode support members.

Each of the electrodes may be electrically connected to an electrical conductor which extends through one of the electrode support members. One, or each, of the electrodes and its associated conductor may be formed from a single wire. The electrical conductor may be enclosed within the electrode support member. The electrode may be exposed for contact with a subject.

Each of the electrode support members may support a single electrode, or a plurality of electrodes.

The probe may further comprise a shaft. The shaft may comprise a tube having a side wall with a plurality of lumens extending along the side wall. Each of the electrode supporting members may extend into a respective one of the lumens. Each of the electrode supporting members may extend along the length of the shaft.

The tube may have a central lumen. The plurality of lumens may be helical, extending around the central lumen and along the length of the shaft.

The invention further provides a method of producing a multi-electrode surgical probe, wherein the probe comprises a basket and a plurality of electrodes supported by the basket, and the basket comprises a plurality of composite fibres each comprising a flexible electrode support member and a conductor embedded in the support member, the method comprising providing a preform of polymeric material with a channel extending through it, inserting a conducting wire into the channel, heating the preform, and drawing the wire through the heated preform whereby the polymeric material coats the wire and forms one of the electrode support members.

The heating may melt a part of the preform. The melted part of the preform may be drawn with the wire. The melted part of the preform may narrow around the wire as it is drawn so as to coat the wire.

The method may further comprise removing an area of the polymer material so as to expose a part of the wire, thereby forming an electrode.

The method may further comprise processing a part of the electrode support member to form a hinge.

The method may further comprise providing a multi-lumen tube having a central lumen surrounded by a side wall, and a plurality of lumens formed in the side wall, and inserting a part of each of the composite fibres into a respective one of the plurality if lumens. The plurality if lumens may be helical, extending around the central lumen as well as along the length of the multi-lumen tube.

The probe may further comprise, in any workable combination, any one or more further features of the embodiments of the invention as will now be described in more detail with reference to the accompanying drawings.

### BRIEF DESCRIPTION OF THE DRAWINGS

FIG. 1a is a perspective view of a probe according to an embodiment of the invention in a contracted configuration;

FIG. 1b is a perspective view of the probe of FIG. 1a in a partially deployed state;

FIG. 1c is a perspective view of the probe of FIG. 1a in a fully deployed state;

FIG. 2a shows detail of the outer basket of the probe of FIG. 1a in the contracted configuration;

FIG. 2b is a perspective view of the outer basket of FIG. 2a in a partially deployed state;

FIG. 2c is a perspective view of the outer basket of FIG. 2a in a fully deployed state;

FIG. 3a shows detail of the inner basket of the probe of FIG. 1a in the contracted configuration;

FIG. 3b is a perspective view of the inner basket of FIG. 3a in a partially deployed state;

FIG. 3c is a perspective view of the inner basket of FIG. 3a in a fully deployed state;

FIG. 4a is an exploded view of the components of the probe of FIG. 1a;

FIGS. 4b to 4d show the components of FIG. 4a at different points during assembly of the probe;

FIG. 5 is a perspective view of an apparatus for producing parts of the probe of FIG. 1a;

FIG. 6 shows detail of part of the apparatus of FIG. 5;

FIG. 7 illustrates operation of the apparatus of FIG. 5;

FIG. 7a shows detail of part of FIG. 7;

FIG. 8 is a cross section through a polymer pre-form used in the apparatus of FIG. 5;

FIG. 9 is a cross section through a flexible member formed from the preform of FIG. 8;

FIG. 10 is a longitudinal section through the preform of FIG. 9 with encapsulated wires during drawing;

FIG. 10a shows detail of part of FIG. 10;

FIG. 11 illustrates a method of drawing a multi-lumen tubing of the probe of FIG. 1a;

FIG. 12 illustrates the shape of one of the lumens in the tubing of FIG. 11;

FIG. 13 illustrates the shape of one of the lumens in an alternative embodiment;

FIG. 14 shows how the embodiment of FIG. 13 is formed;

FIG. 15 shows part of a probe according to a further embodiment of the invention;

FIG. 15a is an enlargement of part A of FIG. 15;

FIGS. 16 to 21 show the basket of a further embodiment of the invention in various states of deployment, from contracted to fully deployed.

### DETAILED DESCRIPTION

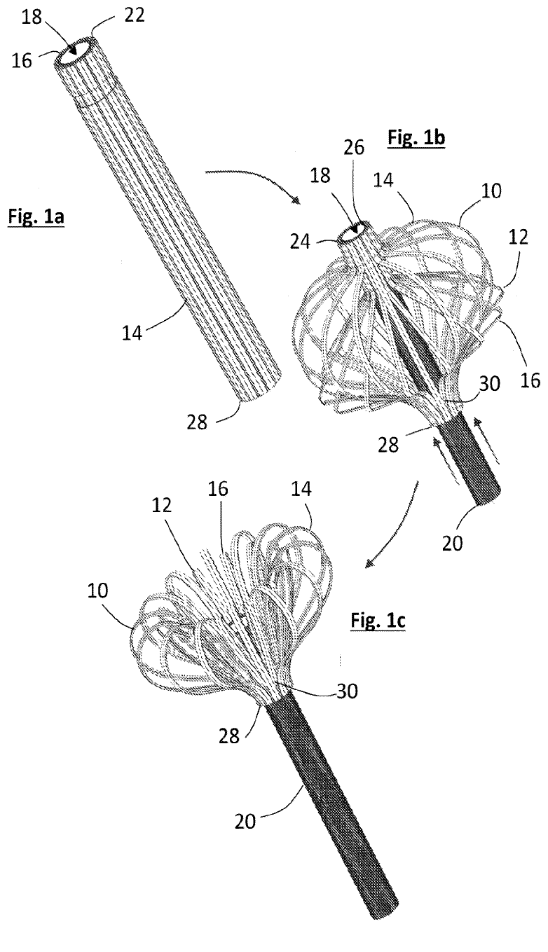

Referring to FIGS. 1a to 1c a multi-electrode surgical probe comprises a flexible basket 10 supporting electrodes, as will be described below. The basket 10 is flexible between a contracted configuration, as shown in FIG. 1a, a partially deployed configuration as shown in FIG. 1b, and a deployed configuration as shown in FIG. 1c. The probe further comprises a bi-stable structure 12 which is stable in both the deployed configuration and the contracted configuration. The basket 10 is formed of a number of flexible electrode support members 14. The bistable structure 12 may be in the form of a further basket, for example an inner basket, and may also comprise a number of flexible bistable members 16, which may be formed of Nitinol or other alloys or materials with similar properties of flexibility and resilience.

The configuration of the flexible members 14, 16 may take various forms, but as shown in FIG. 1a, when the probe is in the contracted configuration the flexible electrode support members 14 may be in the form of straight splines all extending parallel to each other along the length of the probe, and spaced around the central lumen 18 of the probe to form a generally tubular structure. The flexible bistable members 16 may also be in the form of straight splines all extending parallel to each other along the length of the probe, and spaced around the central lumen 18 of the probe to form a generally tubular structure. The bistable members 16 may be located inside the electrode support members 14. A central tube 20 may be located within the tubular structures of the electrode supporting members 14 and the bistable members 16, defining the central lumen 18. This central lumen 18 is open on both the retracted and deployed states of the probe, and can be used to introduce tools or implements through the probe while the probe is in use. The probe may therefore form the end of a catheter through which, for example, ablation tools can be inserted.

At the distal end 22 of the probe, the ends 24 of the electrode support members 14, and the ends 26 of the bistable members 16 are supported and retained in position around the end of the central tube 20. The proximal ends 28 of the electrode support members 14 and the proximal ends 30 of the bistable members 14 are fixed relative to each other, but are slidable along the central tube 20 towards and away from the distal end 22.

To move the probe from the contracted configuration of FIG. 1a, the proximal ends of the electrode supporting members 14 are pushed towards the distal end 22 of the probe, so that the electrode supporting members 14 flex outwards as shown in FIG. 1b so as to form an approximately spherical structure, and then flex further as shown in FIG. 1c so as to form an approximately toroidal structure with their proximal ends 28 close to their distal ends 24. At the same time, the proximal ends 30 of the bistable members 16 move towards their distal ends 26 and the bistable members flex out of their stable contracted configuration, through an unstable partially deployed configuration as shown in FIG. 1b, and then to a stable deployed configuration as shown in FIG. 1c. The bistable members 16 thus resist movement of the probe out of the retracted configuration of FIG. 1a, and also retain the electrode supporting members 14 in their deployed configuration once that configuration is reached, resisting movement of the probe out of the deployed configuration.

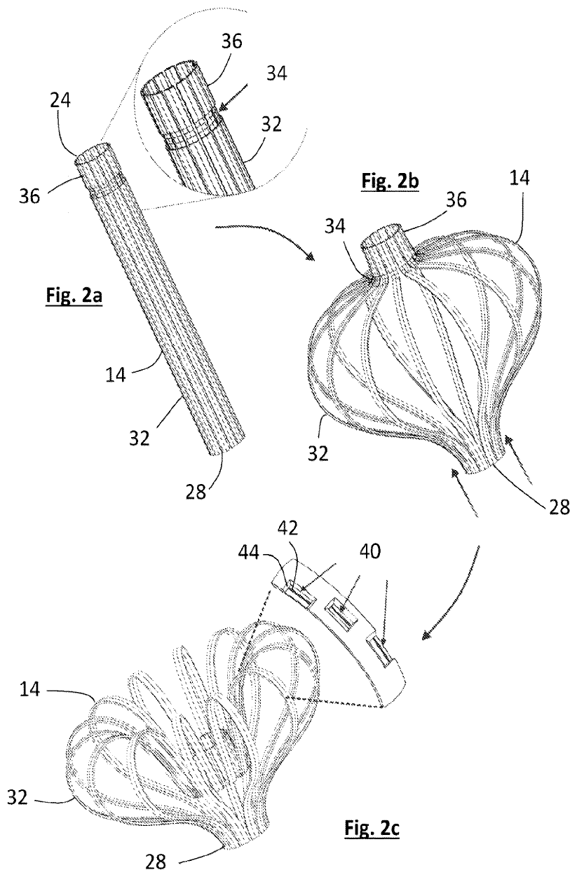

Referring to FIG. 2a the flexing of the electrode support members 14 may be controlled by shaping the members 14. For example, each of the members 14 may be of constant cross section along a main part 32 of its length, but may have a reduced thickness hinge portion 34 formed in it so as to increase the ease with which it flexes at that hinge portion 34. For example, as shown in FIG. 2a, the hinge portion 34 may be located close to the distal end 24 of the members 14. The distal end portion 36 of the member 14 beyond the hinge portion 34 may then form a support portion which is arranged to be supported at the distal end of the probe. The main portion 38 of the member 14, to the proximal side of the hinge portion 34, can therefore flex outwards by substantial flexing of the hinge portion 34 while the support portion remains substantially straight, and the main portion 38 flexes less than the hinge portion 34. If there is no hinge portion formed at the proximal end 28 of the members 14, as the proximal ends 28 are moved towards the distal ends 24, as shown in FIG. 2b the degree of flexing at the proximal ends 28 is less than in the hinge portions 34, and so the main portions 38 flex outwards and forwards over the distal end portions 36 until the fully deployed configuration is reached, in which the basket formed by the main portions 38 extends forwards beyond the distal ends 24 of the members 14 as shown in FIG. 2c. Each of the electrode supporting members 14 supports at least one electrode 40, and a conductor 42 connected to the electrode and extending along the member 14 towards its proximal end 28. The conductor 42 may be embedded within the member 14, which is made of electrically non-conductive material and therefore insulates it from the conductors in the other members. The electrodes 40 may be formed by simply forming an opening 44 in the material of the member 14 to expose a portion of the conductor 42. Optionally each of the openings 44 may be filled with conducting material so as to form a lager electrode having an outer surface which is flush with the surface of the electrode support member 14. As shown in FIG. 2c, each member 14 may support more than one electrode, for example three, each with its respective conductor 42.

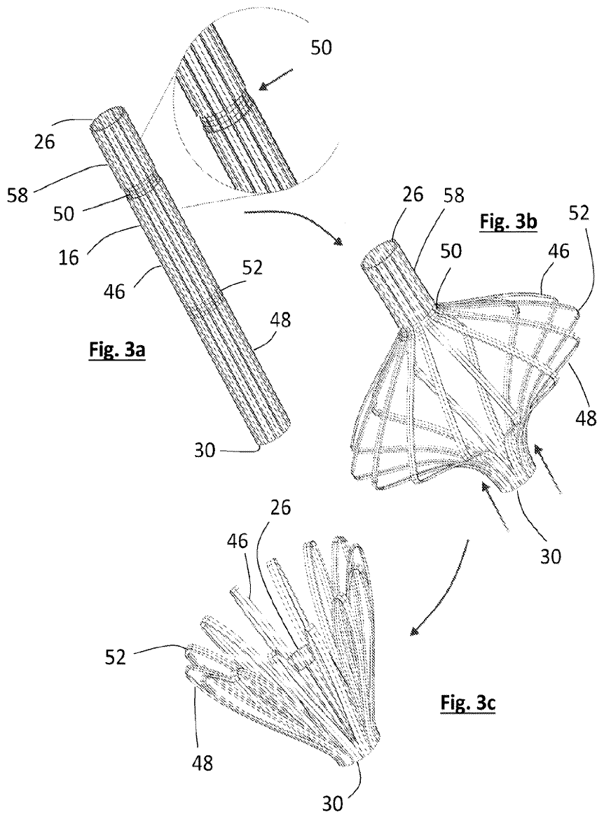

Referring to FIGS. 3a, 3b and 3c the flexing of the bistable members 16 may also be controlled by shaping the members 16. For example, each of the members 16 may be of constant cross section along two main parts 46, 48 of its length, but may have two reduced thickness hinge portions 50, 52 formed in it so as to increase the ease with which it flexes at that hinge portions 50, 52. For example, as shown in FIG. 3a, one of the hinge portions 50 may be located close to the distal end 26 of the members 16, and one of the hinge portions 52 may be located between the first hinge portion 50 and the proximal end 30 of the member 16 to form the two main portions 46, 48 on opposite sides of the second hinge portion 52. The distal end portion 58 of the member 16 beyond the hinge portion 50 may then form a support portion which is arranged to be supported at the distal end of the probe. The main portions 46, 48 of the member 16, can therefore flex outwards as the proximal ends 30 are moved towards the distal ends 26. The more distal main portion 46 can fold outwards relatively easily due to the flexibility of the more distal hinge portion 50. However the more proximal main portion 46 is flexed outwards as shown in FIG. 3b, which provides resistance to movement of the proximal ends 30 towards the distal ends. There is a tipping point when the more distal main portion 46 is extending radially outwards, and so the flexing of the proximal main portions 48 is at a maximum. On further movement of the proximal ends 30 towards the distal ends 26, the more distal main portions 46 start to fold inwards, and so the more proximal main portions 48 can also flex inwards, reducing their flexing as they return towards a straight configuration, where the probe is in its deployed configuration. This urges the members towards a stable folded condition as shown in FIG. 3c. The exact position of the deployed configuration will depend mainly on the balance between the forces from the flexing of the more proximal main portions 48 of the bistable members 16, and the forces from the flexing of the electrode support members 14. However, once the probe has been assembled, with the electrode support members 14 and the bistable members 16 connected to each other at their distal and proximal ends as shown in FIGS. 1a to 1c, the deployed configuration will be fixed, and not dependent on any additional forces applied to the probe.

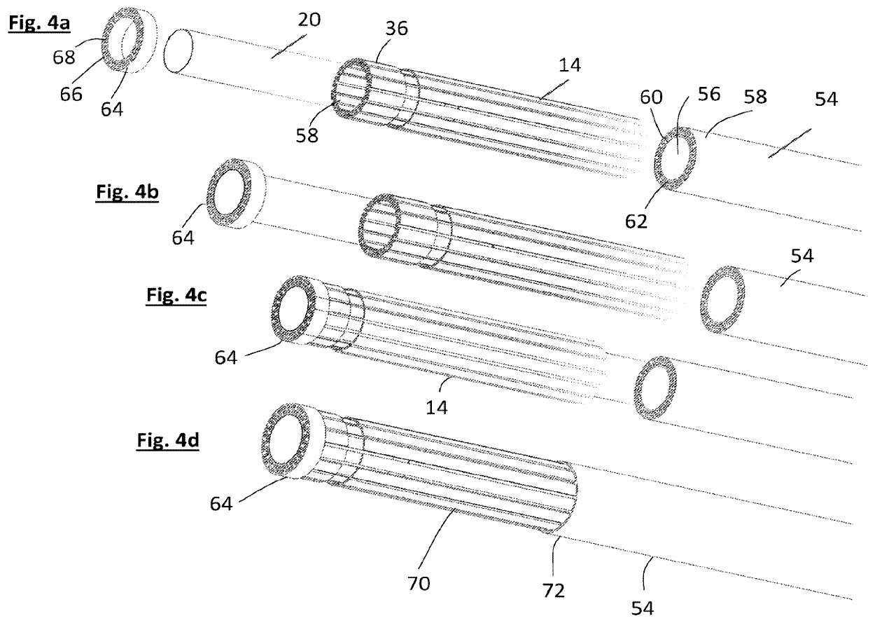

Referring to FIG. 4a, the proximal ends of the electrode supporting members 14 may be supported in a multi-lumen tube 54. The multi-lumen tube may have a central lumen 56 in which the central tube 20 is located, and an annular section side wall 58 having an outer ring 60 of lumens and an inner ring 62 of lumens. The outer ring of lumens 60 are each arranged to house the proximal end 28 of one of the electrode support members 14. In order to provide electrical connection to the electrodes 40, the proximal ends 28 of the electrode supporting members may extend through the whole length of the multi-lumen tube 54. A distal spline support 64 may be mounted on the distal end of the central tube 20. The distal spline support 64 may have an outer ring of support apertures 66 and an inner ring of support apertures 68, in which the short sections of the outer and inner rings of lumens are arranged to support the support sections 36, 58 at the distal ends of the electrode support members 14 and the bistable members 16. The distal spline support 64 may conveniently be formed from a section of the same tubing as the multi-lumen tube 54. The distal spline support 64 may be mounted on the end of the central tube 20 as shown in FIG. 4b, and then the distal ends of the electrode supporting members 14 and the bistable members 16 inserted into the spline support 64 as shown in FIG. 4c.

Once the probe is assembled as shown in FIG. 4d, there is a fixed exposed length 70 of the electrode support members 14 and the bistable members 16 extending between the distal end 72 of the multi-lumen tube 54 and the distal spline support 64. The central tube 20 may be slidable within the multi-lumen tube 54 so as to move the distal spline support 64 towards the end 72 of the multi-lumen tube 54 to move the probe from the retracted configuration shown in FIG. 4d to the deployed configuration as described above.

It will be appreciated that other embodiments can differ in a variety of ways from the embodiments shown. For example the number and shape of both the electrode support members 14 and the bistable members 16 may be different. Indeed the electrode support members may themselves be bistable, though this can result in a less stable configuration of the electrode array. While the bistable members 16 form part of the basket structure of the probe in the embodiments shown, they may be separate from the basket, and may for example take other forms. For example there may be just one or two bistable members similar to those described above, but thicker so that each of them provides more force than in the embodiment shown. Alternatively the bistable members could take a different form altogether.

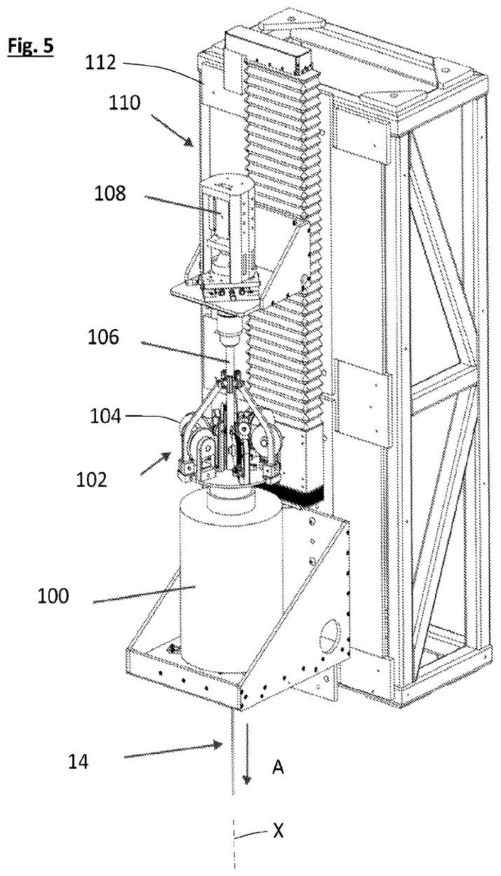

Referring to FIG. 5, the electrode support elements 14 may be formed by a number of suitable methods, but one suitable method is a drawing process in which the conducting elements 42, which are typically in the form of electrically conducting wires, are drawn through a preform of the electrically non-conducting polymeric material of the electrode support element 14, and the preform is heated and drawn into the narrow cross section of the electrode support element 14, with the conducting elements embedded within it. The apparatus for performing this process may comprise a heater or furnace 100 in which the polymeric preform is held and heated, and a wire feed system 102 from which the conducting elements 42 are fed into the furnace 100 from spools 104. In general terms, the electrode support element 14 is drawn from the furnace 100 in the direction of the arrow A, as the electrode support element 14 has the conducting elements 42 embedded in it, that also pulls the conducting elements 42 from the spools 104. The polymeric preform within the furnace 100 is melted and drawn out with the conducting elements forming a coating around them.

The wire feed system 102 may be rotatably mounted so that it can rotate about a central axis X that is aligned with the drawn electrode support element 14. The preform, which will be described in more detail below, may be support on a preform holder 106 that extends through the wire feed system 102 along the same axis X. The preform holder may also be rotatable about the same axis X. A single spinning motor 108 may be connected to both the preform holder 106 and optionally also to the wire feed platform 102 so that it can rotate one or both of them about the axis X during the drawing process. The spinning motor 108 may only be used during formation of the multi-lumen tube 54 as will be described below. The furnace 100, wire feed platform 102, preform holder 106, and motor 108 may all be supported on a suitable structure such as a tower 112.

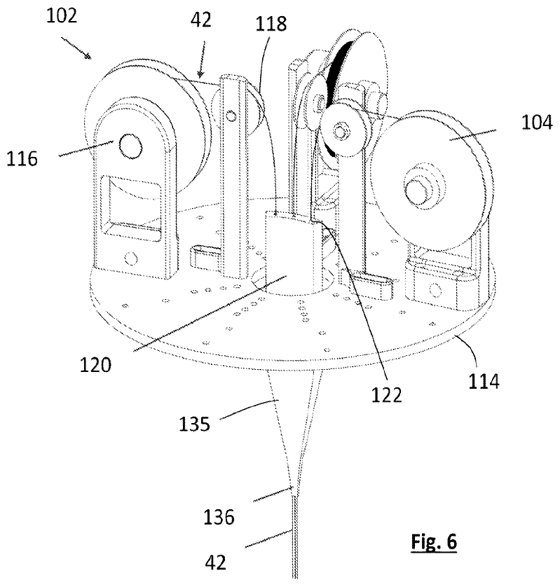

Referring to FIG. 6, the wire feed system 102 may comprise a rotatably mounted platform 114 on which the spools 104 are mounted via respective spool supports 116, optionally together with guide rollers 118 which guide the conducting elements 42 from the spools 104 into the preform. The preform 120 is supported in the centre of the wire feed system on its axis of rotation X. The preform 120 may have a number of channels 122 formed through it into each of which a respective one of the conducting elements 42 is fed.

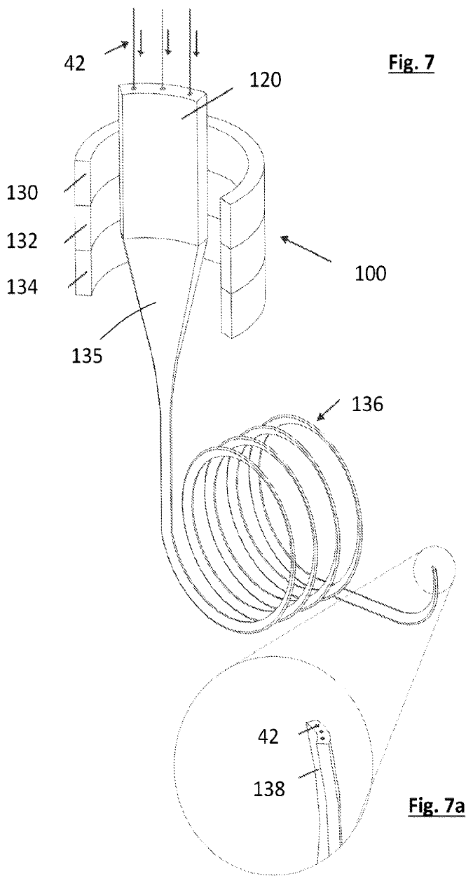

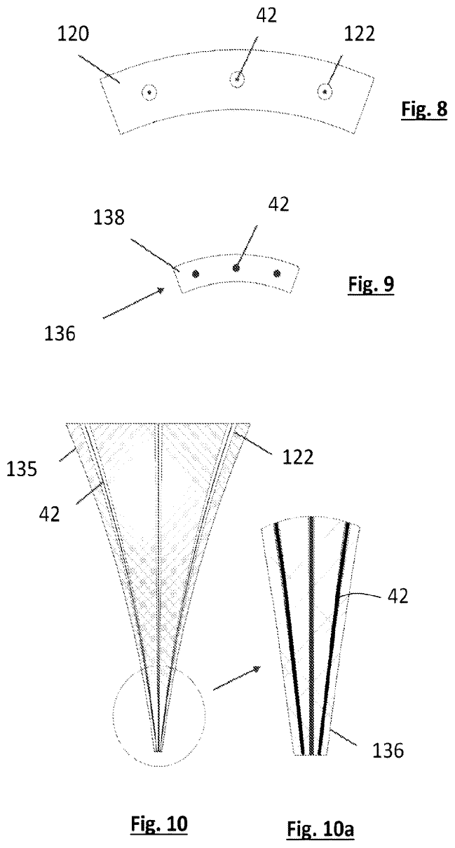

Referring to FIG. 7, in operation, preform 120 is held within the furnace 100, which may include a number of heating zones 130, 132, 134. The channels 122 extend through the preform 120 parallel to each other and parallel to the draw axis X. At this point where the preform 120 is still solid and un-melted, the channels 122 are larger in diameter than the conducting elements 42 which therefore extend loosely through them, as shown in FIG. 8, and can be drawn through them. The drawn polymeric material 135 from the preform 120 with the conducting elements 42 embedded within it form a continuous composite fibre 136 which can be pulled away from the furnace 100 to continue the drawing process. That pulling draws melted polymer from the preform 120, causing the melted part of the preform to narrow down so that it forms a polymeric coating 138 of the composite fibre 136, and is significantly narrower than the preform 120. However the composite fibre retains the cross sectional shape of the preform 120. Therefore the preform 120 may be of substantially rectangular cross section so that the composite fibre 136 is also of substantially rectangular cross section. At the same time the conducting elements 42 are pulled through the preform 120, and the channels shrink around them until the polymeric material coats the surface of the conducting elements in the composite fibre 136 as shown in FIGS. 10 and 10a. However the conducting elements 42 remain separated by the polymeric coating 138 so that each of them is insulated from the others within the composite fibre 136 as shown in FIG. 9.

In order to form the electrode supporting members 14 from the composite fibre, the fibre 136 is cut into lengths, the hinges 34 are formed by cutting a way a portion of the fibre 126 to reduce its thickness, preferably without exposing the conducting elements 42. The openings 44 are then cut into the fibre 126 to expose regions of the conducting elements 42 thereby forming the electrodes 40. The rectangular cross section of the composite fibre 136 means that multiple electrodes can be formed on one flat side of the electrode supporting members 14 as shown in FIG. 2c. It will be appreciated that other cross sections could be used but if more than one electrode is supported on each of the electrode supporting members 14 then it is preferable for the cross section to be such that there is a single flat or substantially flat surface which can be cut away to form all of the electrodes.

The bistable members 16 do not need to include the conducting elements 42, but may be formed form a drawing process similar to that described for the electrode supporting members 14, but using solid preforms with no channels 122 through them, and without the conducting elements being fed into the drawing process.

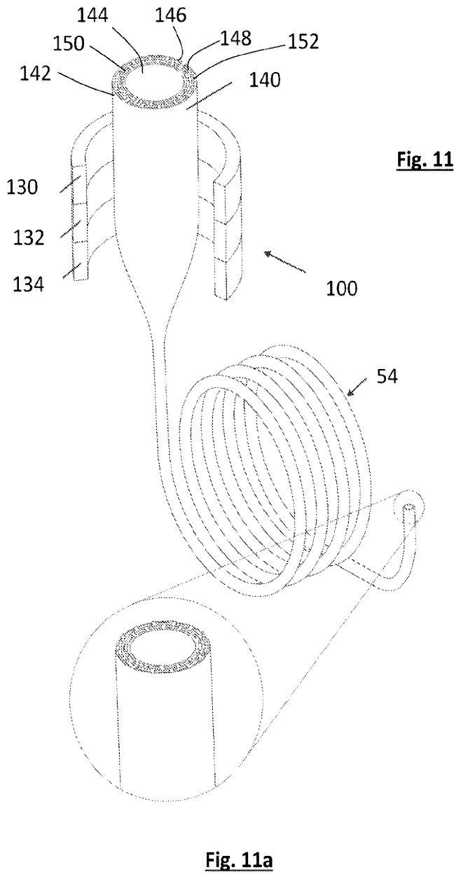

Referring to FIG. 11, the multi-lumen tube 54 may be formed in a similar manner to the to the bistable members 16. In this case the preform 140 has the same general cross section as the multi-lumen tube 54, but with a much larger diameter. It therefore comprises a generally tubular member 142 having a central opening or through bore 144, and an annular section side wall 146. The lumens 148 are formed in the side wall 146, in an inner ring 150 and an outer ring 152. The preform 140 is heated in the furnace 100 and drawing out to form the multi-lumen tube 54.

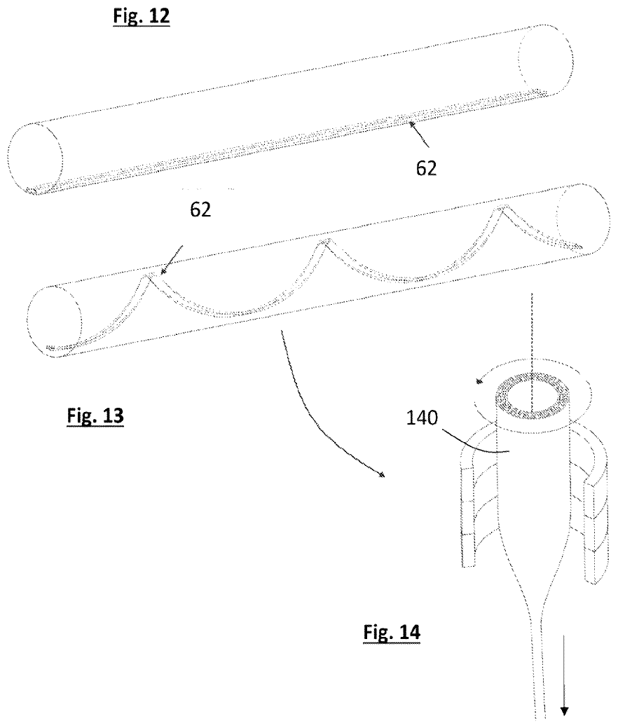

Referring to FIG. 12, if the multi-lumen tube 54 is drawn from the preform 140 without use of the spinning motor 108, the lumens 62, 64 for the electrode supporting members 14 are straight and parallel to the central axis of the tube 54. However, referring to FIG. 14, if the spinning motor 108 is operated to rotate the preform 140 during drawing of the multi-lumen tube 54, then the lumens 62, 64 are spiral in form as shown in FIG. 13, extending around the central lumen 54 as well as along the length of the probe shaft. Therefore when the electrode supporting members 14 are inserted into the lumens 62, 64, the conducting elements 42 are also in a spiral configuration. This has the advantage that the shaft of the finished probe, which comprises the multi-lumen tube 54 with the electrode supporting members and the conducting elements 42 extending through it, is more flexible than in the configuration of FIG. 11, in which the wire conducting elements, which are generally inextensible, prevent easy bending of the probe shaft.

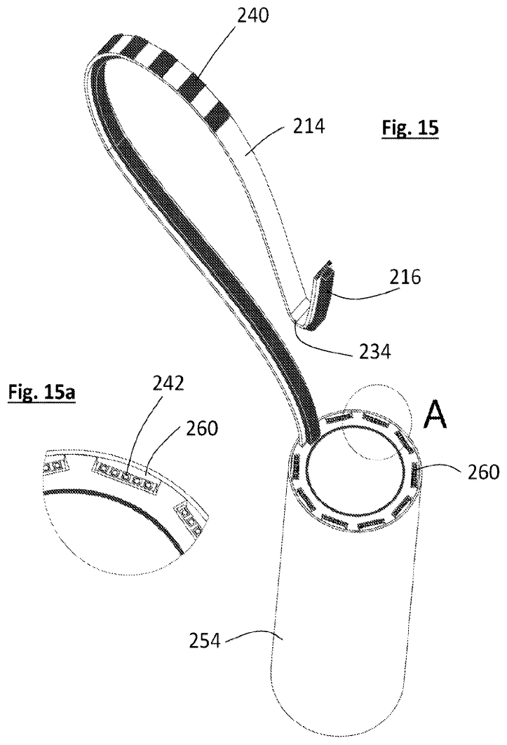

Referring to FIG. 15, in a further embodiment, the electrode support members 214 are themselves of a similar structure to those 14 of FIG. 2c, each comprising an elastomeric support with conducting elements 242 extending through it and connected to electrodes 240. However the bistable structure is achieved by attaching one or more flexible stiffening members 216 to each of the electrode support members 214. The basket is therefore formed of a number of identical flexible members or splines each of which supports a number of electrodes 240 and each of which is bistable. The bistable structure may be achieved by having stiffening members 216 connected to parts of the conducting elements 242 to form stiffer regions, and parts 234 of the conducting elements having no stiffening material connected to them so that they are more flexible and can form hinge portions.

The electrode supporting elements 214 may be supported in a multi-lumen tube 254, and may have their distal ends supported in a spline support, which is movable by means of a central tube in the same way as the embodiment of FIGS. 4a to 4d. However because the electrode support elements 214 themselves form the bistable structure, there is no need for separate lumens in the multi-lumen tube 254 for the separate bistable structure, and so all of the lumens 260 in the multi-lumen tube 254 are used for electrode support elements 214. As can be seen in FIG. 15, and in FIG. 15a, the stiffening members 216 do not need to extend into the multi-lumen tube 254, but are attached to the surface, conveniently the inner surface, of the electrode supporting elements 214 between the end of the multi-lumen tube and the spline support. In FIG. 15 only one of the composite bistable electrode supporting members is shown which is in the fully deployed condition. The central tube and distal spline support are also not shown.

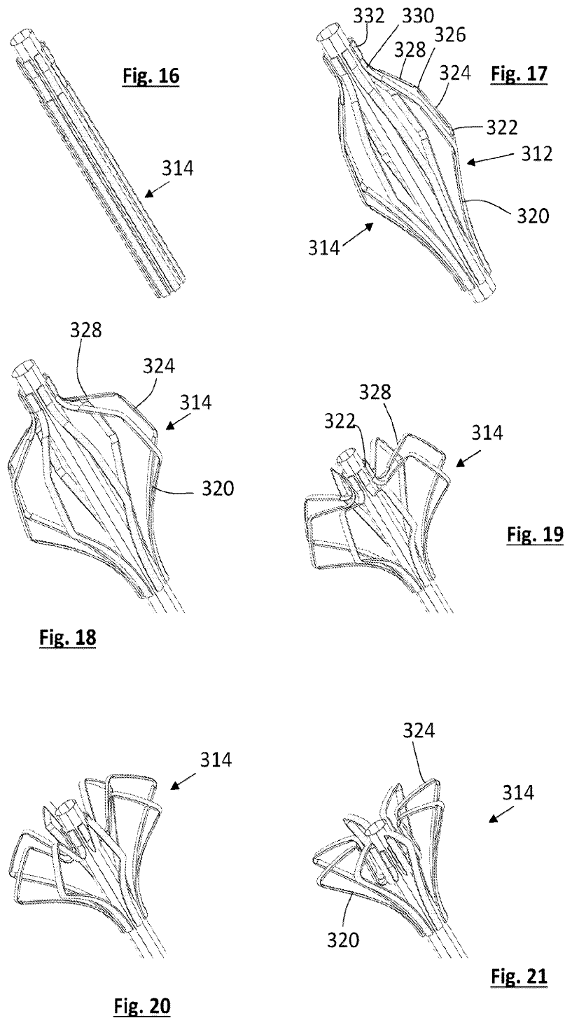

Referring to FIGS. 16 to 21, in a further embodiment, the structure of the basket 312 is similar to that of FIG. 15, except for the length and location of the stiffening members 316. In this case each of the bistable electrode supporting members 314 comprises, from the proximal end where it is supported in the multi-lumen tube, a first stiffened portion 320 to which a stiffening member 316 formed form a length of stiffening material is connected, a first hinge portion 322, at the distal end of the first stiffened portion 320, to which no stiffening material is attached, then a second stiffened portion 324, then a second hinge portion 326, then a third stiffened portion 328, then a third hinge portion 330, and finally a distal support portion 332 which is supported on the distal end of the central tube 340, for example by means of a spline support.

In operation, from the fully retracted condition as shown in FIG. 16 in which the splines are all parallel and as the distal support portions 332 of the splines are moved down towards the proximal end, the first stiffened portions 320 and the third hinge portions 330 bend outwards, until over-centre position which is approximately as shown in FIG. 19 is reached in which the third stiffened portions 328 extend approximately radially outwards. On further movement of the distal support portions 322 the third stiffened portions 328 fold inwards towards the distal support portions 322, the first stiffened portions 320 also flex back inwards, and the second stiffened portions move towards the distal end of the probe. As shown in FIG. 21, in the fully deployed condition, the third hinge portions 330 are folded through 180° so that the third stiffened portions 328 are approximately parallel to each other and the axis of the probe, and the second stiffened portions extend approximately radially outwards. In this condition the flexing of the first stiffened portions 320 pushes the third stiffened portions inwards into contact with the distal support portions 322, which prevents further movement and therefore defines the fully deployed condition. To start movement back towards the contracted condition, the distal support portions 322 are moved back towards the distal end of the probe causing folding of the third stiffened portions 328 outwards, which pushes the first stiffened portions 320 outwards causing them to flex back outwards towards their over-centre position.

All of the electrodes may be located on the second stiffened portion 324 such that, when the basket is in the fully deployed condition, the electrodes are approximately in a single plane perpendicular to the axis of the probe.

1. A multi-electrode surgical probe comprising a basket, and a plurality of electrodes supported on the basket, wherein the basket is flexible between a contracted configuration and a deployed configuration, and the probe comprises a bi-stable structure which is stable in both the deployed configuration and the contracted configuration.

2. A probe according to claim 1 wherein the basket comprises a plurality of flexible electrode support members each of the electrode support members supporting at least one of the electrodes, and the electrode support members are arranged to flex between a first shape and a second shape as the basket flexes between the contracted configuration and the deployed configuration; wherein the first shape of the electrode support members is straight and the second shape is curved.

3. A probe according to claim 2 wherein when the electrode support members form a hollow tubular structure when the probe is in the contracted configuration.

4. A probe according to claim 1 wherein the basket has a first end and a second end and the probe further comprises first and second supports each supporting a respective one of the first and second ends of the basket.

5. A probe according to claim 4 wherein each of the first and second supports is tubular defining an opening therethrough, the probe further comprises an instrument channel through which an instrument can be moved through the probe, and the openings surround the instrument channel.

6. A probe according to claim 4 wherein the first support is movable relative to the second support thereby to flex the basket between the contracted configuration and the deployed configuration.

7. A probe according to claim 1 wherein the probe comprises a bistable member which is arranged to resist movement of the probe out of both the contracted configuration and the deployed configuration; wherein the bistable member is a flexible member having a folded state and an unfolded state; and wherein the bistable member comprises first and second main portions and a hinge portion connecting the first and second main portions to each other.

8. A probe according to claim 7 wherein the bistable member further comprises a support portion and a further hinge portion connecting the support portion to the second main portions.

9. A probe according to claim 8 wherein the probe further comprises first and second supports, the first main portion has a first end which is supported in the first support, and the support portion is supported in the second support, and the first and second supports are moveable relative to each other to move the bistable member between its folded and unfolded states.

10. A probe according to claim 7 wherein the bistable member further comprises a second hinge portion, a third main portion connected to the second main portion by the second hinge portion, and a support portion connected to the third main portion by a third hinge portion.

11. A probe according to claim 10 wherein the probe has an axis, and in the deployed configuration, the second main portion extends radially outwards from the axis of the probe.

12. A probe according to claim 10 wherein the electrodes are supported on the second main portion of the bistable member.

13. A probe according to claim 7 wherein the bistable structure comprises a plurality of bistable members.

14. A probe according to claim 7 wherein the basket comprises a plurality of flexible electrode support members each of the electrode support members supporting at least one of the electrodes, and the electrode support members are arranged to flex between a first shape and a second shape as the basket flexes between the contracted configuration and the deployed configuration, and wherein, when the probe is in the contracted state, the bistable member is parallel to the electrode support members.

15. A probe according to claim 1 further comprising a plurality of electrical conductors each of which extends through a respective one of the electrode support members, wherein each of the electrodes is electrically connected to one of the electrical conductors.

16. A probe according to claim 15 wherein one of the electrodes and its associated electrical conductor are formed from a single wire, the electrical conductor being enclosed within the electrode support member and the electrode being exposed for contact with a subject.

17. A probe according to claim 1 wherein the probe further comprises a shaft, and the shaft comprise a tube having a side wall with a plurality of lumens extending along the side wall, and each of the electrode supporting members extends into a respective one of the lumens and along a length of the shaft.

18. A probe according to claim 17 wherein the tube has a central lumen, and the plurality of lumens are helical extending around the central lumen and along the length of the shaft.

19. A method of producing a multi-electrode surgical probe, wherein the probe comprises a basket and a plurality of electrodes supported by the basket, wherein the basket comprises a plurality of composite fibres each comprising a flexible electrode support member and a conductor embedded in the support member, the method comprising providing a preform of polymeric material with a channel extending through it, inserting a conducting wire into the channel, heating the preform, and drawing the wire through the heated preform whereby the polymeric material coats the wire and forms one of the electrode support members.

20. A method according to claim 19 further comprising removing an area of the polymer material so as to expose a part of the wire, whereby the exposed part of the wire forms an electrode; processing a part of the electrode support member to form a hinge; and providing a multi-lumen tube having a central lumen surrounded by a side wall, and a plurality of lumens formed in the side wall, and inserting a part of each of the composite fibres into a respective one of the plurality if lumens; wherein the plurality if lumens are helical, extending around the central lumen as well as along the length of the multi-lumen tube.
